(19) United States
(12) Patent Application Publication
(10) Pub. No.: US 2011/0053983 A1
(43) Pub. Date: Mar. 3, 2011

(54) (5R)-1,5-DIARYL-4,5-DIHYDRO-1H-PYRAZOLE-3-CARBOXAMIDINE DERIVATIVES HAVING CB1-ANTAGONISTIC ACTIVITY

(76) Inventors: Josephus H.M. Lange, CP Weesp (NL); Arnold P. Den Hartog, CP Weesp (NL); Bernard J. Van Vliet, CP Weesp (NL)

(21) Appl. No.: 12/988,654

(22) PCT Filed: Apr. 22, 2009
(86) PCT No.: PCT/EP09/54788

§ 371 Date: Nov. 16, 2010

Related U.S. Application Data
(60) Provisional application No. 61/047,251, filed on Apr. 23, 2008.

(30) Foreign Application Priority Data
Apr. 23, 2008 (EP) 08155009.7

(51) Int. Cl. A61K 31/454; C07D 401/12; A61P 25/00; A61P 3/04; A61P 1/00; A61P 3/10; A61P 11/06; A61P 19/02; A61P 9/00; A61P 35/00

(52) U.S. Cl. 514/326; 546/211

(57) ABSTRACT

Embodiments of the invention relate to (5R)-1,5-diaryl-4,5-dihydro-1H-pyrazole-3-carboxamidine derivatives as cannabinoid-CB₁ receptor antagonists, to methods for the preparation of these compounds, to novel intermediates useful for the synthesis of said dihydropyrazole derivatives, to methods for the preparation of these intermediates, pharmaceutical compositions containing one or more of these dihydropyrazole derivatives as an active ingredient, as well as to the use of these pharmaceutical compositions for the treatment of psychiatric and neurological disorders involving cannabinoid receptors. The compounds of embodiments of the invention are compounds of formula (I) wherein the symbols have the meanings given in the specification.

This invention relates to the fields of pharmaceutical and organic chemistry, and provides (5R)-1,5-diaryl-4,5-dihydro-1H-pyrazole-3-carboxamidines, intermediates, formulations and methods.

### BACKGROUND

SR141716A, now known as rimonabant, and other CB₁ receptor modulators, including CB₁/CB₂ receptor subtype selective receptor antagonists, have several potential therapeutic applications such as medicaments for treating psychosis, anxiety, depression, attention deficits, memory disorders, cognitive disorders, appetite disorders, obesity, addiction, drug dependence, neurodegenerative disorders, dementia, dystonia, muscle spasticity, tremor, epilepsy, multiple sclerosis, traumatic brain injury, stroke, Parkinson's disease, Alzheimer's disease, epilepsy, Huntington's disease, Tourette's syndrome, cerebral ischaemia, cerebral apoplexy, craniocerebral trauma, stroke, spinal cord injury, neuroinflammatory disorders, plaque sclerosis, viral encephalitis, demyelinisation related disorders, pain disorders, neuropathic pain disorders, septic shock, glaucoma, diabetes, cancer, emesis, nausea, gastrointestinal disorders, gastric ulcers, diarrhoea, sexual disorders, impulse control disorders and cardiovascular disorders (Boyd, 2005; Sorbera, 2005; Carai, 2005; Lange, 2004 & 2005; Hertzog, 2004; Smith, 2005; Thakur, 2005; Padgett, 2005; Muccioli, 2005 & 2006; Reggio, 2003; Adam, 2006; Högenauer, 2007).

EP 1 713 475 (first published as WO 2005/074920) disclosed racemic 1,3,5-trisubstituted 4,5-dihydro-1 H-pyrazole derivatives as CB₁ receptor antagonists, including N-[(piperidin-1-yl)-sulfonyl]-N′-methyl-1-(4-chlorophenyl)-5-phenyl-4,5-dihydro-(1H)-pyrazole-3-carboxamidine:

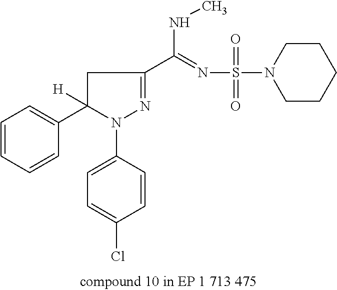

This compound however, as well as the other compounds disclosed, were found inactive (ID₅₀>30 mg/kg) in the in vivo CB₁ mediated (CP-55,940-induced) hypotension test after oral administration (see below). The present invention aims at the development of 1,3,5-trisubstituted 4,5-dihydro-1 H-pyrazole-derived CB₁ receptor antagonists or inverse agonists with improved in vivo activities after oral administration.

### DISCLOSURE

Potent and selective antagonism or inverse agonism of cannabinoid-CB₁ receptors was found in (5R)-1,5-diaryl-4,5-dihydro-1H-pyrazole-3-carboxamidine derivatives of formula (I):

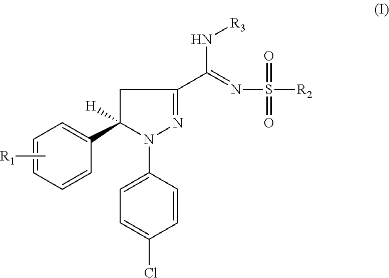

or a tautomer, stereoisomer, N-oxide, or a pharmacologically acceptable salt, of any of the foregoing, wherein:
R₁ is a hydrogen or a fluoro atom,
R₂ represents a piperidinyl or a pyrrolidinyl group, optionally group is substituted with one or two fluoro atoms or a trifluoromethyl group,
R₃ is a methyl or ethyl group

Further embodiments provide one or more a (5R)-enantiomers of compounds of formula (I) wherein: R₂ is a piperidin-1-yl group, substituted on its 4-position with one or two fluoro atoms or a trifluoromethyl group, and R₃ is a methyl group.

The invention also relates, in some embodiments, to a compound of formula (I*):

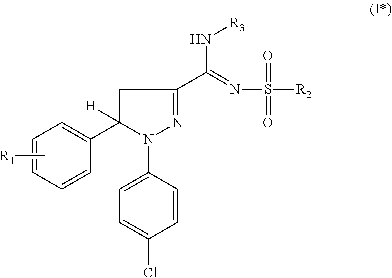

wherein the symbols have the meanings as given above, chosen from:
N-[(4,4-difluoropiperidin-1-yl)sulfonyl]-N′-methyl-1-(4-chlorophenyl)-5-phenyl-4,5-dihydro-(1H)-pyrazole-3-carboxamidine
N-[(4,4-difluoropiperidin-1-yl)sulfonyl]-N′-methyl-1-(4-chlorophenyl)-5-(2-fluorophenyl)-4,5-dihydro-(1H)-pyrazole-3-carboxamidine.
N-[(4-(trifluoromethyl)piperidin-1-yl)sulfonyl]-N′-methyl-1-(4-chlorophenyl)-5-phenyl-4,5-dihydro-(1H)-pyrazole-3-carboxamidine
N-[(4-fluoropiperidin-1-yl)sulfonyl]-N′-methyl-1-(4-chlorophenyl)-5-phenyl-4,5-dihydro-(1H)-pyrazole-3-carboxamidine
N-[(3-fluoropiperidin-1-yl)sulfonyl]-N′-methyl-1-(4-chlorophenyl)-5-phenyl-4,5-dihydro-(1H)-pyrazole-3-carboxamidine
N-[(3,3-difluoropiperidin-1-yl)sulfonyl]-N′-methyl-1-(4-chlorophenyl)-5-phenyl-4,5-dihydro-(1H)-pyrazole-3-carboxamidine
N-[(3,3-difluoropyrrolidin-1-yl)sulfonyl]-N′-methyl-1-(4-chlorophenyl)-5-phenyl-4,5-dihydro-(1H)-pyrazole-3-carboxamidine
N-[(4,4-difluoropiperidin-1-yl)sulfonyl]-N′-ethyl-1-(4-chlorophenyl)-5-phenyl-4,5-dihydro-(1H)-pyrazole-3-carboxamidine
N-[(4-fluoropiperidin-1-yl)sulfonyl]-N′-ethyl-1-(4-chlorophenyl)-5-phenyl-4,5-dihydro-(1H)-pyrazole-3-carboxamidine
   such compounds being useful in the preparation of compounds of formula (I).

The compounds of the invention of the formula (I), as well as the pharmacologically acceptable salts thereof, have cannabinoid CB₁ receptor modulating activity. They are useful in the treatment of disorders involving cannabinoid receptors, or treatable via manipulation of those receptors. The compounds of the invention have considerably higher CB₁ receptor affinities and higher CB₁ antagonistic potencies than their corresponding (5S)-counterparts. In addition, some of the compounds of the invention are active in CB₁ receptor mediated in vivo models after oral administration. The compounds of the invention can be brought into forms suitable for administration by means of usual processes using auxiliary substances and/or liquid or solid carrier materials.

Other embodiments of the invention include:

pharmaceutical compositions for treating, for example, a disorder or condition that may be treated by modulating cannabinoid CB₁ receptors, the compositions comprising a compound of formula (I) or a pharmaceutically acceptable salt thereof, and a pharmaceutically acceptable carrier;

methods of treatment of a disorder or condition that may be treated by modulating cannabinoid CB₁ receptors, the methods comprising administering to a patient in need of such treatment a compound of formula (I) or a pharmaceutically acceptable salt thereof;

pharmaceutical compositions for treating, for example, a disorder or condition chosen from psychosis, anxiety, depression, attention deficits, memory disorders, cognitive disorders, appetite disorders, obesity, addiction, drug dependence, neurodegenerative disorders, dementia, dystonia, muscle spasticity, tremor, multiple sclerosis, traumatic brain injury, stroke, Parkinson's disease, Alzheimer's disease, epilepsy, Huntington's disease, Tourette's syndrome, cerebral ischaemia, cerebral apoplexy, craniocerebral trauma, stroke, spinal cord injury, neuroinflammatory disorders, plaque sclerosis, viral encephalitis, demyelinisation related disorders, pain disorders, chronic pain, neuropathic pain, acute pain and inflammatory pain, osteoporosis, septic shock, glaucoma, diabetes, emesis, nausea, gastrointestinal disorders, gastric ulcers, diarrhoea, sexual disorders, impulse control disorders and cardiovascular disorders;

methods of treatment of a disorder or condition chosen from the disorders listed herein, the methods comprising administering to a patient in need of such treatment a compound of formula (I) or a pharmaceutically acceptable salt thereof;

pharmaceutical compositions for treatment of a disorder or condition chosen from the disorders listed herein, the compositions comprising a compound of formula (I) or a pharmaceutically acceptable salt thereof, and a pharmaceutically acceptable carrier;

The invention also provides the use of a compound or salt according to formula (I) for the manufacture of a medicament.

The invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed. Such other therapeutic agent(s) may be administered prior to, simultaneously with, or following the administration of the compounds of the invention.

The invention also provides compounds, pharmaceutical compositions, kits and methods for the treatment of a disorder or condition that may be treated by modulating cannabinoid CB₁ receptors, the method comprising administering to a patient in need of such treatment a compound of formula (I) or a pharmaceutically acceptable salt thereof.

The invention also provides methods of preparing the compounds of the invention and the intermediates used in those methods.

Isolation and purification of the compounds and intermediates described herein can be affected, if desired, by any suitable separation or purification procedure such as, for example, filtration, extraction, crystallization, column chromatography, thin-layer chromatography, thick-layer chromatography, preparative low or high-pressure liquid chromatography, or a combination of these procedures. Specific illustrations of suitable separation and isolation procedures can be taken from the preparations and examples. However, other equivalent separation or isolation procedures could, of course, also be used.

Some of the crystalline forms for the compounds may exist as polymorphs, and as such are intended to be included in the present invention. In addition, some of the compounds may form solvates with water (i.e., hydrates) or common organic solvents, and such solvates are also intended to be encompassed within the scope of this invention.

Isotopically-labeled compound of formula (I) or pharmaceutically acceptable salts thereof, including compounds of formula (I) isotopically-labeled to be detectable by PET or SPECT, are also included within the scope of the invention, and same applies to compounds of formula (I) labeled with [¹³C], [¹⁴C]-, [³H]-, [¹⁸F]-, [¹²⁵]- or other isotopically enriched atoms, suitable for receptor binding or metabolism studies.

### Definitions

General terms used in the description of compounds herein disclosed bear their usual meanings. The term “substituted” means that the specified group or moiety bears one or more substituents. Where any group may carry multiple substituents, and a variety of possible substituents is provided, the substituents are independently selected, and need not to be the same. The term “unsubstituted” means that the specified group bears no substituents. To provide a more concise description, the terms ‘compound’ or ‘compounds’ include tautomers, stereoisomers, N-oxides, isotopically-labelled analogues, or pharmacologically acceptable salts, hydrates or solvates, also when not explicitly mentioned.

N-oxides of the compounds mentioned above belong to the invention. Tertiary amines may or may not give rise to N-oxide metabolites. The extent to what N-oxidation takes place varies from trace amounts to a near quantitative conversion. N-oxides may be more active than their corresponding tertiary amines, or less active. Whilst N-oxides can easily be reduced to their corresponding tertiary amines by chemical means, in the human body this happens to varying degrees. Some N-oxides undergo nearly quantitative reductive conversion to the corresponding tertiary amines, in other cases conversion is a mere trace reaction, or even completely absent (Bickel, 1969).

‘Crystal form’ refers to various solid forms of the same compound, for example polymorphs, solvates and amorphous forms. ‘Polymorphs’ are crystal structures wherein a compound can crystallize in different crystal packing arrangements, all having the same elemental composition. Polymorphism is a frequently occurring phenomenon, affected by several crystallization conditions such as temperature, level of supersaturation, the presence of impurities, polarity of solvent, rate of cooling. Different polymorphs usually have different X-ray diffraction patterns, solid state NMR spectra, infrared or Raman spectra, melting points, density, hardness, crystal shape, optical and electrical properties, stability, and solubility. Recrystallization solvent, rate of crystallization, storage temperature, and other factors may cause one crystal form to dominate. ‘Solvates’ are generally crystal forms containing either stoichiometric or non-stoichiometric amounts of a solvent. Often, during the process of crystallization some compounds have a tendency to trap a fixed molar ratio of solvent molecules in the crystalline solid state, thus forming a solvate. When the solvate is water, ‘hydrates’ may be formed. The compound of formula (I) and pharmaceutically acceptable salts thereof may exist in the form of a hydrate or a solvate, and such a hydrate and solvate are also encompassed in the present invention. Examples thereof include ¼ hydrate, dihydrochloride dihydrate, etc. ‘Amorphous’ forms are noncrystalline materials with no long range order, and generally do not give a distinctive powder X-ray diffraction pattern. Crystal forms in general have been described by Byrn (1995) and Martin (1995)

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to experimental or measurement conditions for such given value.

Throughout the description and the claims of this specification the word “comprise” and variations of the word, such as “comprising” and “comprises” is not intended to exclude other additives, components, integers or steps.

While it may be possible for the compounds of formula (I) to be administered as the raw chemical, it is preferable to present them as a ‘pharmaceutical composition’. According to a further aspect, the present invention provides a pharmaceutical composition comprising at least one compound of formula (I), at least one pharmaceutically acceptable salt or solvate thereof, or a mixture of any of the foregoing, together with one or more pharmaceutically acceptable carriers thereof, and optionally one or more other therapeutic ingredients. The carrier(s) must be ‘acceptable’ in the sense of being compatible with the other ingredients of the formulation and not deleterious to the recipient thereof. The term “composition” as used herein encompasses a product comprising specified ingredients in predetermined amounts or proportions, as well as any product resulting, directly or indirectly, from combining specified ingredients in specified amounts. In relation to pharmaceutical compositions, this term encompasses a product comprising one or more active ingredients, and an optional carrier comprising inert ingredients, as well as any product resulting, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. The pharmaceutical composition includes enough of the active object compound to produce the desired effect upon the progress or condition of diseases. Accordingly, the pharmaceutical compositions of the present invention encompass any composition made by mixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

Within the context of this application, the term ‘combination preparation’ comprises both true combinations, meaning a compound of formula (I) and other medicaments physically combined in one preparation such as a tablet or injection fluid, as well as ‘kit-of-parts’, comprising a compound of formula (I) and another medicament in separate dosage forms, together with instructions for use, optionally with further means for facilitating compliance with the administration of the component compounds, e.g. label or drawings. With true combinations, the pharmacotherapy by definition is simultaneous. The contents of ‘kit-of-parts’, can be administered either simultaneously or at different time intervals. Therapy being either concomitant or sequential will be dependant on the characteristics of the other medicaments used, characteristics like onset and duration of action, plasma levels, clearance, etc., as well as on the disease, its stage, and characteristics of the individual patient.

The affinity of the compounds of the invention for cannabinoid CB₁ receptors was determined as described below. From the binding affinity measured for a given compound of formula (I), one can estimate a theoretical lowest effective dose. At a concentration of the compound equal to twice the measured Kᵢ-value, nearly 100% of the cannabinoid CB₁ receptors likely will be occupied by the compound. By converting that concentration to mg of compound per kg of patient—assuming ideal bioavailability—a theoretical lowest effective dose is obtained. Pharmacokinetic, pharmacodynamic, and other considerations may alter the dose actually administered to a higher or lower value. The dose of the compound to be administered will depend on the relevant indication, the age, weight and sex of the patient and may be determined by a physician. The dosage will preferably be in the range of from 0.01 mg/kg to 10 mg/kg. The typical daily dose of the active ingredients varies within a wide range and will depend on various factors such as the relevant indication, the route of administration, the age, weight and sex of the patient and may be determined by a physician. In general, total daily dose administration to a patient in single or individual doses, may be in amounts, for example, from 0.001 to 10 mg/kg body weight daily, and more usually from 0.01 to 1,000 mg per day, of total active ingredients. Such dosages will be administered to a patient in need of treatment from one to three times each day, or as often as needed for efficacy, and for periods of at least two months, more typically for at least six months, or chronically.

The term “therapeutically effective amount” as used herein refers to an amount of a therapeutic agent to treat a condition treatable by administrating a composition of the invention. That amount is the amount sufficient to exhibit a detectable therapeutic or ameliorative response in a tissue system, animal or human. The effect may include, for example, treating the conditions listed herein. The precise effective amount for a subject will depend upon the subject's size and health, the nature and extent of the condition being treated, recommendations of the treating physician, and the therapeutics, or combination of therapeutics, selected for administration. Thus, it is not useful to specify an exact effective amount in advance. The term “pharmaceutically acceptable salt” refers to those salts, within the scope of sound medical judgment, suitable for use in contact with the tissues of humans without undue toxicity, irritation, allergic response, etc., and are commensurate with a reasonable benefit/risk ratio. Pharmaceutically acceptable salts are well-known in the art. They can be prepared in situ when finally isolating and purifying the compounds of the invention, or separately by reacting them with pharmaceutically acceptable non-toxic bases or acids, including inorganic or organic bases and inorganic or organic acids (Berge, 1977). The ‘free base’ form may be regenerated by contacting the salt with a base or acid, and isolating the parent compound in the conventional matter. The parent form of the compound differs from the various salt forms in certain physical properties, such as solubility in polar solvents, but otherwise the salts are equivalent to the parent form of the compound for the purposes of the present invention. The term “treatment” refers to any treatment of a human condition or disease, and includes: (1) inhibiting the disease or condition, i.e., arresting its development, (2) relieving the disease or condition, i.e., causing the condition to regress, or (3) stopping the symptoms of the disease. The term ‘inhibit’ includes its generally accepted meaning, including restraining, alleviating, ameliorating, and slowing, stopping or reversing progression, severity, or a resultant symptom. As used herein, the term “medical therapy” intendeds to include diagnostic and therapeutic regimens carried out in vivo or ex vivo on humans.

As used herein ‘obesity’ refers to a condition whereby a person has a Body Mass Index (BMI), calculated as weight per height squared (km/m²), of at least 25.9. Conventionally, those persons with normal weight have a BMI of 19.9 to less than 25.9. The obesity herein may be due to any cause, whether genetic of environmental. Examples of disorders that may result in obesity or be the cause of obesity include overeating and bulimia, polycystic ovarian disease, craniopharyngioma, the Prader-Willi syndrome, Frohlich's syndrome, Type-II diabetes, GH-deficient subjects, normal variant short stature, Turners syndrome, and other pathological conditions showing reduced metabolic activity or a decrease in resting energy expenditure as a percentage of total fat-free mass, e.g. children with acute lymphoblastic leukemia.

### EXAMPLE 1

### GENERAL ASPECTS OF SYNTHESES

The synthesis of compounds having formula (I) is outlined in Scheme 1:

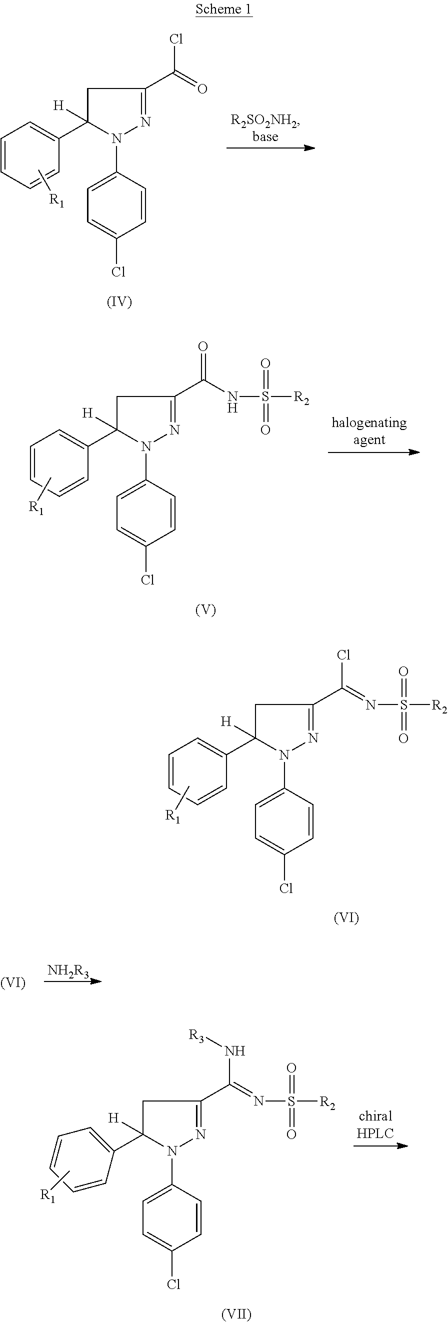

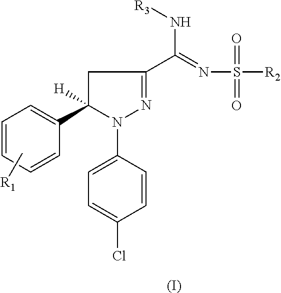

A carbonyl chloride of formula (IV) can be reacted with a compound of formula R₂SO₂NH₂ in the presence of a base such NaH or NaOH, to give a compound of formula (V) wherein R₁ and R₂ have the abovementioned meaning. The compound of formula (V) can be reacted with a halogenating agent, for example a chlorinating agent such as POCl₃ to give a compound of formula (VI). Such a reaction is preferably carried out in the presence of 4-dimethylaminopyridine (DMAP). Compound (VI) can be reacted with an amine of formula NH₂R₃ to give a compound of formula (VII), wherein R₁, R₂ and R₃ have the meaning as given above. Compound (VII) can be separated via chiral preparative HPLC to give compound (I), wherein R₁, R₂ and R₃ have the meaning as given above and wherein C₅ of its 4,5-dihydropyrazole moiety has the R configuration.

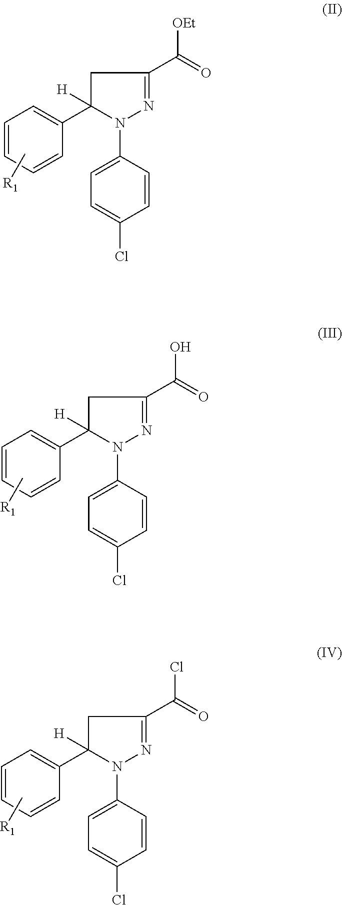

Intermediates with formulae (II), (Ill) or (IV) were obtained according to published methods (EP 1 713 475, WO 2005/077911, EP 1 743 892, Srivastava, 2007).
Compounds described below were prepared according to these procedures. They are intended to further illustrate the invention in more detail, not to restrict the scope of the invention in any way. Other embodiments of the invention will be apparent to those skilled in the art from consideration of the specification and practice of the invention disclosed herein. The specification and examples must be considered as exemplary only.

The selection of the particular synthetic procedures depends on factors known to those skilled in the art such as the compatibility of functional groups with the reagents used, the possibility to use protecting groups, catalysts, activating and coupling reagents and the ultimate structural features present in the final compound being prepared.

Pharmaceutically acceptable salts may be obtained using procedures well-known in the art, for example by mixing a compound of the present invention with a suitable acid, for instance an inorganic acid or an organic acid.

### EXAMPLE 2

### SYNTHESES OF SPECIFIC COMPOUNDS

### (5R)-(+)-N-[(4,4-difluoropiperidin-1-yl)sulfonyl]-N′-methyl-1-(4-chlorophenyl)-5-phenyl-4,5-dihydro-(1H)-pyrazole-3-carboxamidine (compound 1) and (5S)-(−)-N-[(4,4-difluoropiperidin-1-yl)sulfonyl]-N′-methyl-1-(4-chlorophenyl)-5-phenyl-4,5-dihydro-(1H)-pyrazole-3-carboxamidine (compound 2)

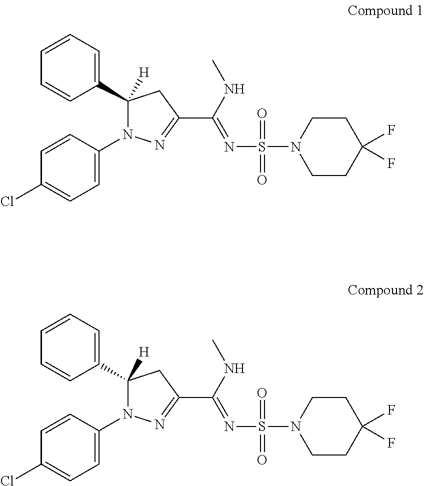

Step 1: Sulfamide (9.15 g; 95.2 mmol) was added to 4,4-difluoropiperidine hydrochloride (15.0 g; 95.2 mmol) in butyl acetate (200 ml). DIPEA (17.9 ml; 104.7 mmol) was added and the magnetically stirred reaction mixture was heated at reflux temperature overnight. The reaction mixture was allowed to attain room temperature. Volatiles were removed in vacuo. Ethyl acetate and 1N HCl were successively added. The organic layer was separated and dried over Na₂SO₄. After filtration, the filtrate was collected and volatiles were removed in vacuo. The product was washed twice with diisopropyl ether affording 4,4-difluoropiperidin-lylsulfonamide (15.96 g; 83.8%). ¹H-NMR (400 MHz, DMSO-d₆): δ 2.02-2.14 (m, 4H); 3.10-3.16 (m, 4H), 6.90 (br s, 2H). All ¹H NMR spectra disclosed herein, were recorded on a Bruker 400 MHz instrument using CDCl₃ or DMSO-d₆ as solvent with tetramethylsilane as an internal standard. Chemical shifts are given in ppm (δ scale) downfield from tetramethylsilane. Coupling constants (J) are expressed in Hz.

Step 2: To 1-(4-chlorophenyl)-5-phenyl-4,5-dihydro-(1H)-pyrazole-3-carboxylic acid (18.77 g; 62.4 mmol, prepared as described in EP 1 713 475) in toluene (200 ml) was added thionyl chloride (18.00 ml; 246.8 mmol). The reaction mixture was stirred at 80° C. for 1 hour. Volatiles were removed in vacuo. 50 ml toluene was added and again volatiles were removed in vacuo. The formed 1-(4-chlorophenyl)-5-phenyl-4,5-dihydro-(1H)-pyrazole-3-carboxylic acid chloride was dissolved in 250 ml acetonitrile: solution A. To a solution of 4,4-difluoropiperidine-1-sulfonamide (12.50 g; 62.4 mmol) in 500 ml acetonitrile was added aqueous NaOH (8.25 ml; 157.79 mmol). After 10 minutes solution A was slowly added. The reaction mixture was stirred overnight at room temperature. Volatiles were removed in vacuo to give crude N-[(4,4-difluoropiperidin-1-yl)sulfonyl]-1-(4-chlorophenyl)-5-phenyl-4,5-dihydro-(1H)-pyrazole-3-carboxamide (39.01 g). This crude residue was extracted with CH₂Cl₂/1N HCl. Layers were separated. The dichloromethane layer was sucessively dried with Na₂SO₄, filtered and evaporated affording N-[(4,4-difluoropiperidin-1-yl)sulfonyl]-1-(4-chlorophenyl)-5-phenyl-4,5-dihydro-(1H)-pyrazole-3-carboxamide (30.41 g, quantitative yield). ¹H-NMR (400 MHz, DMSO-d₆): δ 1.93-2.10 (m, 4H); 2.75 (dd, J=18 and 6 Hz, 1H), 3.12-3.21 (m, 4H), 3.36 (br s, probably contains NH and H₂O), 3.62 (dd, J=18 and 13 Hz, 1H), 5.42 (dd, J=13 and 6 Hz, 1H), 6.93 (br d, J=8, 2H), 7.14-7.36 (m, 7H).

Step 3: N-[(4,4-Difluoropiperidin-1-yl)sulfonyl]-1-(4-chlorophenyl)-5-phenyl-4,5-dihydro-(1H)-pyrazole-3-carboxamide (30.14 g; 62.4 mmol) was dissolved in dichloromethane (500 ml). DMAP (33.80 g; 276.7 mmol) was added. POCl₃ (phosphorus oxychloride) (7.35 ml; 80.3 mmol) in dichloromethane (50.00 ml) was added dropwise. The reaction mixture was refluxed for 4 hours. After cooling down to 6° C. methylamine hydrochloride(19.0 g; 281.4 mmol) was added, followed by dropwise addition of DIPEA (72.0 ml; 420.6 mmol). The reaction mixture was stirred overnight at room temperature. Water (100 ml) was added, followed by acidification with 1N hydrochloric acid. Layers were separated. The dichloromethane layer was dried over Na₂SO₄, filtered and concentrated in vacuo. Purification by flash chromatography on silicagel (silica gel 60 (0.040-0.063 mm, Merck, used for all flash chromatography disclosed herein) (diethyl ether/petroleum ether (40/60)=1/1 (v/v)) afforded N-[(4,4-difluoropiperidin-1-yl)sulfonyl]-N′-methyl-1-(4-chlorophenyl)-5-phenyl-4,5-dihydro-(1H)-pyrazole-3-carboxamidine (27.1 g; 87.6%, racemate 1). ¹H-NMR (400 MHz, DMSO-d₆): δ 2.00-2.15 (m, 4H); 2.85 (br d, J˜5, 3H), 3.09-3.21 (m, 5H), 3.94 (dd, J=18 and 13 Hz, 1H), 5.61 (dd, J=13 and 6 Hz, 1H), 7.04 (br d, J=8, 2H), 7.20-7.38 (m, 7H), 8.80-8.85 m, 1H).

Analogously were prepared:

N-[(4,4-difluoropiperidin-1-yl)sulfonyl]-N′-methyl-1-(4-chlorophenyl)-5-(2-fluorophenyl)-4,5-dihydro-(1H)-pyrazole-3-carboxamidine (racemate 2). Melting point: 166-167° C. (recorded on a Büchi 8-545 melting point apparatus, as were all melting points disclosed here).

N-[(4-(trifluoromethyl)piperidin-1-yl)sulfonyl]-N′-methyl-1-(4-chlorophenyl)-5-phenyl-4,5-dihydro-(1H)-pyrazole-3-carboxamidine (racemate 3). ¹H-NMR (400 MHz, DMSO-d₆): δ 1.43-1.60 (m, 2H), 1.83-1.92 (m, 2H), 2.39-2.48 (m, 1H), 2.57-2.69 (m, 2H), 2.88 (br s, 3H), 3.14 (dd, J=17.8, 6.6 Hz, 1H), 3.60 (t, J=9.2 Hz, 2H), 3.94 (dd, J=18.1, 12.9 Hz, 1H), 5.60 (dd, J=12.6, 6.6 Hz, 1H), 7.03 (d, J=9.0 Hz, 2H), 7.23 (d, J=9.0 Hz, 2H), 7.26-7.39 (m, 5H), 8.86 (br s, 1H). Melting point: 172-173° C.

N-[(4-fluoropiperidin-1-yl)sulfonyl]-N′-methyl-1-(4-chlorophenyl)-5-phenyl-4,5-dihydro-(1H)-pyrazole-3-carboxamidine (racemate 4). ¹H-NMR (400 MHz, DMSO-d₆): δ 1.73-2.02 (m, 4H), 2.86 (s, 3H), 3.00-3.23 (m, 5H), 3.94 (dd, J=17.9, 12.8 Hz, 1H), 4.80 (dd, J=48.22, 2.7 Hz, 1H), 5.59 (dd, J=12.8, 6.5 Hz, 1H), 7.03 (br d, J=8.7 Hz, 2H), 7.20-7.38 (m, 7H), 8.86 (br s, 1H).

N-[(3-fluoropiperidin-1-yl)sulfonyl]-N′-methyl-1-(4-chlorophenyl)-5-phenyl-4,5-dihydro-(1H)-pyrazole-3-carboxamidine (racemate 5). ¹H-NMR (400 MHz, DMSO-d₆): δ 1.49-1.84 (m, 4H), 2.87 (d, J=4.5 Hz, 3H), 3.04-3.26 (m, 4H), 3.35-3.41 (m, 1H), 3.88-4.01 (m, 1H), 4.67-4.91 (m, 1H), 5.60 (dd, J=12.8, 6.5 Hz, 1H), 7.03 (br d, J=9.0 Hz, 2H), 7.22 (br d, J=9.0 Hz, 2H), 7.25-7.38 (m, 5H), 8.86 (d, J=4.8 Hz, 1H).

N-[(3,3-difluoropiperidin-1-yl)sulfonyl]-N′-methyl-1-(4-chlorophenyl)-5-phenyl-4,5-dihydro-(1H)-pyrazole-3-carboxamidine (racemate 6). ¹H-NMR (400 MHz, DMSO-d₆): δ 1.68-1.78 (m, 2H), 1.90-2.07 (m, 2H), 2.89 (s, 3H), 3.05 (t, J=5.0 Hz, 2H), 3.13 (dd, J=17.8, 6.6 Hz, 1H), 3.24 (t, J=11.8 Hz, 2H), 3.93 (dd, J=17.9, 12.8 Hz, 1H), 5.62 (dd, J=12.8, 6.5 Hz, 1H), 7.04 (br d, J=9.0 Hz, 2H), 7.22 (br d, J=8.7 Hz, 2H), 7.26-7.39 (m, 5H), 8.94 (br s, 1H).

N-[(3,3-difluoropyrrolidin-1-yl)sulfonyl]-N′-methyl-1-(4-chlorophenyl)-5-phenyl-4,5-dihydro-(1H)-pyrazole-3-carboxamidine (racemate 7). ¹H-NMR (400 MHz, DMSO-d₆): δ 2.36-2.47 (m, 2H), 2.87 (s, 3H), 3.13 (dd, J=17.9, 6.5 Hz, 1H), 3.37 (t, J=7.2 Hz, 2H), 3.54 (t, J=13.7 Hz, 2H), 3.92 (dd, J=17.8, 12.9 Hz, 1H), 5.62 (dd, J=12.8, 6.5 Hz, 1H), 7.04 (br d, J=8.7 Hz, 2H), 7.23 (br d, J=9.0 Hz, 2H), 7.25-7.38 (m, 5H), 8.95 (br s, 1H).

N-[(4,4-difluoropiperidin-1-yl)sulfonyl]-N′-ethyl-1-(4-chlorophenyl)-5-phenyl-4,5-dihydro-(1H)-pyrazole-3-carboxamidine (racemate 8). ¹H-NMR (400 MHz, CDCl₃): δ 1.28-1.43 (m, 3H), 1.98-2.20 (m, 4H), 3.09 (dd, J=18, 7.2 Hz) and 3.40 (dd, J=18.5, 7.7 Hz) (both signals together integrate for 1H), 3.23-3.37 (m, 4H), 3.47-3.59 (m) and 3.84-3.99 (m) (both signals together integrate for 2H), 3.75 (dd, J=18, 13.2 Hz) and 4.19 (dd, J=18.2, 13.4 Hz) (both signals together integrate for 1H), 5.23-5.45 (m, 1H), 6.67 (br s) and 7.77 (br s) (both signals together integrate for 1H), 6.82-7.00 (m, 2H), 7.14 (d, J=8.7 Hz, 2H), 7.19 (d, J=7.5 Hz, 2H), 7.27-7.42 (m, 3H).

N-[(4-fluoropiperidin-1-yl)sulfonyl]-N′-ethyl-1-(4-chlorophenyl)-5-phenyl-4,5-dihydro-(1H)-pyrazole-3-carboxamidine (racemate 9). ¹H-NMR (400 MHz, CDCl₃): δ 1.28-1.42 (m, 3H), 1.83-2.07 (m, 4H), 3.05-3.46 (m, 5H), 3.48-3.64 (m) and 3.84-3.95 (m) (both signals together integrate for 2H), 3.78 (dd, J=18.2, 13.1 Hz) and 4.20 (dd, J=18.1, 13.2 Hz, 1H) (both signals together integrate for 1H), 4.67-4.90 (m, 1H), 5.22-5.44 (m, 1H), 6.64 (br s) and 7.82 (br s) (both signals together integrate for 1H), 6.87-6.98 (m, 2H), 7.14 (d, J=8.7 Hz, 2H), 7.19 (d, J=7.2 Hz, 2H), 7.27-7.40 (m, 3H).

These racemates are useful for the preparation of the corresponding (5R)-enantiomers by preparative chiral HPLC analogously to the procedure described under step 4 below.

Step 4: Racemic N-[(4,4-difluoropiperidin-1-yl)sulfonyl]-N′-methyl-1-(4-chlorophenyl)-5-phenyl-4,5-dihydro-(1H)-pyrazole-3-carboxamidine (26.71 g) was separated into both enantiomers by preparative chiral HPLC. This resulted in (5R)-(+)-N-[(4,4-difluoropiperidin-1-yl)sulfonyl]-N′-methyl-1-(4-chlorophenyl)-5-phenyl-4,5-dihydro-(1H)-pyrazole-3-carboxamidine (12.82 g; 37.27%) (first eluting enantiomer) and (5S)-(−)-N-[(4,4-difluoropiperidin-1-yl)sulfonyl]-N′-methyl-1-(4-chlorophenyl)-5-phenyl-4,5-dihydro-(1H)-pyrazole-3-carboxamidine (12.11 g; 35.21%) (second eluting enantiomer). In order to remove remaining aliphatic trace impurities the obtained (5R)-(+)-N-[(4,4-difluoropiperidin-1-yl)sulfonyl]-N′-methyl-1-(4-chlorophenyl)-5-phenyl-4,5-dihydro-(1H)-pyrazole-3-carboxamidine was dissolved in DCM, washed twice with 1N HCl and co-evaporated in the presence of diisopropyl ether, affording (5R)-(+)-N-[(4,4-difluoropiperidin-1-yl)sulfonyl]-N′-methyl-1-(4-chlorophenyl)-5-phenyl-4,5-dihydro-(1H)-pyrazole-3-carboxamidine (Compound 1) (10.5 g; 30.5%). [α²⁵_{D}]=165°, c=1, methanol. This and all other optical rotations disclosed herein, were measured on a Bellingham/Stanley ADP410 polarimeter. Specific rotations ([α²⁵_{D]}) are given as deg/dm, the concentration values are reported as g/100 ml of the specified solvent.

Melting point: 158° C. (recrystallized twice from absolute ethanol). ¹H-NMR (400 MHz, DMSO-d₆): δ 2.00-2.15 (m, 4H); 2.85 (br d, J˜5, 3H), 3.09-3.21 (m, 5H), 3.94 (dd, J=18 and 13 Hz, 1H), 5.61 (dd, J=13 and 6 Hz, 1H), 7.04 (br d, J=8, 2H), 7.20-7.38 (m, 7H), 8.80-8.85 m, 1H). In order to remove remaining aliphatic trace impurities the obtained (5S)-(−)-N-[(4,4-difluoropiperidin-1-yl)sulfonyl]-N′-methyl-1-(4-chlorophenyl)-5-phenyl-4,5-dihydro-(1H)-pyrazole-3-carboxamidine was dissolved in DCM, washed twice with 1N HCl and co-evaporated in the presence of diisopropyl ether, affording (5S)-(−)-N-[(4,4-difluoropiperidin-1-yl)sulfonyl]-N′-methyl-1-(4-chlorophenyl)-5-phenyl-4,5-dihydro-(1H)-pyrazole-3-carboxamidine (Compound 2) (9.7 g; 29.7%). [α²⁵_{D}]=−176°, c=1, methanol. ¹H-NMR (400 MHz, DMSO-d₆): δ 2.00-2.15 (m, 4H); 2.85 (br d, J˜5, 3H), 3.09-3.21 (m, 5H), 3.94 (dd, J=18 and 13 Hz, 1H), 5.61 (dd, J=13 and 6 Hz, 1H), 7.04 (br d, J=8, 2H), 7.20-7.38 (m, 7H), 8.80-8.85 m, 1H).

### X-Ray determination of (5R)-(+)-N-[(4,4-difluoropiperidin-1-yl)sulfonyl]-N′-methyl-1-(4-chlorophenyl)-5-phenyl-4,5-dihydro-(1H)-pyrazole-3-carboxamidine (compound 1)

The analysis was carried out on a block-shaped crystal, cut out of a cluster of crystals of compound 1. X-ray data were collected with a Nonius KappaCCD diffractometer on a rotating anode at a temperature of 150 K. The PLATON program (Spek, 2003) was used for the analysis of the geometry, the illustrations and the validation of the results. The absolute configuration at C5 was determined as R by using the Bijvoet pair analysis. The “Flack x parameter” value amounted to -0.06 ±0.04.

Preparative chiral HPLC method: A 250×76 mm CHIRALPAK® T101 column was used. Methanol/acetonitrile/diethylamine=85/15/0.1 (v/v) was used as the mobile phase. Flow rate: 250 ml/minute. Temperature: 25° C. Detection UV 220 nm

Analytical chiral HPLC method: A 250×4.6 mm CHIRALPAK® IB column was used. n-heptane/ethanol/diethylamine=80/20/0.1 (v/v) was used as the mobile phase. Flow rate: 1 ml/minute. Temperature: 25° C. Detection UV 360 nm.

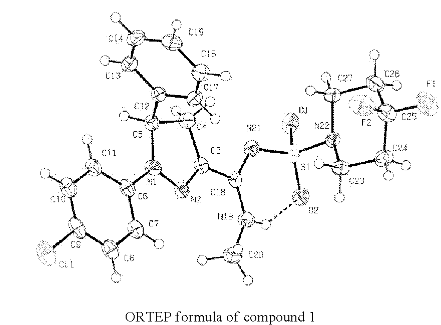

Data of first eluting enantiomer on preparative column: Chemical purity >99% (area% at 360 nm). Enantiomeric excess >99,5%. Data of second eluting enantiomer on preparative column: Chemical purity >99% (area% at 360 nm). Enantiomeric excess >99.5%.

### 5R-(+)-N-[(4-fluoropiperidin-1-yl)sulfonyl]-N′-methyl-1-(4-chlorophenyl)-5-phenyl-4,5-dihydro-(1H)-pyrazole-3-carboxamidine (Compound 3)

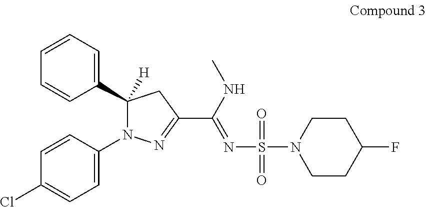

Compound 3 was prepared analogously to compound 1, using 4-fluoropiperidine instead of 4,4-difluoropiperidine. [α²⁵_{D}]=174°, c=1, methanol. ¹H-NMR (400 MHz, DMSO-d₆): δ 1.73-2.02 (m, 4H), 2.86 (s, 3H), 3.00-3.23 (m, 5H), 3.94 (dd, J=17.9, 12.8 Hz, 1H), 4.80 (dd, J=48.2, 2.7 Hz, 1H), 5.59 (dd, J=12.8, 6.5 Hz, 1H), 7.03 (br d, J=8.7 Hz, 2H), 7.20-7.38 (m, 7H), 8.86 (br s, 1H).

Preparative chiral HPLC method: A 250×30 mm CHIRALPAK® AD-H 5 μm column was used. 70/30 Carbon dioxide/Ethanol+1% diethylamine was used as the mobile phase. Flow rate: 120 ml/minute. Temperature: 25° C. Detection UV 300 nm. Outlet pressure: 130 bars.

Analytical chiral HPLC method: A 250×4.6 mm CHIRALPAK® IC 5 μm column was used. n-heptane/ethanol/diethylamine=70/30/0.1 (v/v) was used as the mobile phase. Flow rate: 1 ml/minute. Temperature: 25° C. Detection: Diode array detection (DAD) 230 nm.

Data of compound 3 on preparative column: Chemical purity >99% (area% at 230 nm). Enantiomeric excess >98%. Retention time: 8.07 min.

### 5R-(+)-N-[(4-(trifluoromethyl)piperidin-1-yl)sulfonyl]-N′-methyl-1-(4-chlorophenyl)-5-phenyl-4,5-dihydro-(1H)-pyrazole-3-carboxamidine (Compound 4)

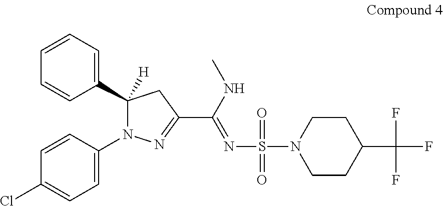

Compound 4 was prepared analogously to compound 1 using 4-(trifluoromethyl)piperidine instead of 4,4-difluoropiperidine. [α²⁵_{D}]=167°, c=1, methanol. ¹H-NMR (400 MHz, DMSO-d₆): δ 1.43-1.60 (m, 2H), 1.83-1.92 (m, 2H), 2.39-2.48 (m, 1H), 2.57-2.69 (m, 2H), 2.88 (br s, 3H), 3.14 (dd, J=17.8, 6.6 Hz, 1H), 3.60 (t, J=9.2 Hz, 2H), 3.94 (dd, J=18.1, 12.9 Hz, 1H), 5.60 (dd, J=12.6, 6.6 Hz, 1H), 7.03 (d, J=9.0 Hz, 2H), 7.23 (d, J=9.0 Hz, 2H), 7.26-7.39 (m, 5H), 8.86 (br s, 1H).

Preparative chiral HPLC method: A 250×30 mm CHIRALPAK® AD-H 5 μm column was used. 70/30 Carbon dioxide/Ethanol+1% diethylamine was used as the mobile phase. Flow rate: 120 ml/minute. Temperature: 25° C. Detection: UV 250 nm. Outlet pressure: 130 bars.

Analytical chiral HPLC method: A 250×4.6 mm CHIRALPAK® IA 5 μm column was used. n-heptane/ethanol/diethylamine=70/30/0.1 (v/v) was used as the mobile phase. Flow rate: 1 ml/minute. Temperature: 25° C. Detection DAD 250 nm.

Data of compound 4 on preparative column: Chemical purity >98% (area% at 250 nm). Enantiomeric excess >98%. Retention time: 7.22 min.

### EXAMPLE 3

### PHARMACOLOGICAL METHODS

In vitro affinity for cannabinoid-CB₁ receptors was determined using membrane preparations of Chinese hamster ovary (CHO) cells wherein the human cannabinoid CB₁ receptor was stably transfected in conjunction with [³H]CP-55,940 as radioligand. After incubation of a freshly prepared cell membrane preparation with the [³H]-ligand, with or without addition of compounds of the invention, separation of bound and free ligand was performed by filtration over glassfiber filters. Radioactivity on the filter was measured by liquid scintillation counting. The binding data were either obtained by CEREP (128, rue Danton, 92500 Rueil-Malmaison, France) or at Solvay Pharmaceuticals B.V. (C. J. van Houtenlaan 36, 1381 C P Weesp, The Netherlands).

In vitro cannabinoid-Ca₁ receptor antagonism was assessed with human CB₁ receptors cloned in Chinese hamster ovary (CHO) cells. CHO cells were grown in a Dulbecco's Modified Eagle's medium (DMEM) culture medium, supplemented with 10% heat-inactivated fetal calf serum. Medium was aspirated and replaced by DMEM, without fetal calf serum, but containing [³H]-arachidonic acid and incubated overnight in a cell culture stove (5% CO₂/95% air; 37° C.; water-saturated atmosphere). During this period [³H]-arachidonic acid was incorporated in membrane phospholipids. On the test day, medium was aspirated and cells were washed three times using 0.5 mL DMEM, containing 0.2% bovine serum albumin (BSA). Stimulation of the CB₁ receptor by WIN 55,212-2 lead to activation of PLA₂ followed by release of [³H]-arachidonic acid into the medium. This WIN 55,212-2-induced release was concentration-dependently antagonized by CB₁ receptor antagonists.

In vivo cannabinoid-CB₁ receptor antagonism was assessed with the CP-55,940-induced hypotension test in rat. Male normotensive rats (225-300 g; Harlan, Horst, The Netherlands) were anaesthetized with pentobarbital (80 mg/kg ip). Blood pressure was measured, via a cannula inserted into the left carotid artery, by means of a Spectramed DTX-plus pressure transducer (Spectramed B. V., Bilthoven, The Netherlands). After amplification by a Nihon Kohden Carrier Amplifier (Type AP-621G; Nihon Kohden B. V., Amsterdam, The Netherlands), the blood pressure signal was registered on a personal computer (Compaq Deskpro 386s), by means of a Po-Ne-Mah data-acquisition program (Po-Ne-Mah Inc., Storrs, USA). Heart rate was derived from the pulsatile pressure signal. All compounds were administered orally as a microsuspension in 1% methylcellulose, 30 minutes before induction of the anesthesia, 60 minutes prior to administration of the CB₁ receptor agonist CP-55,940. The injection volume was 10 ml/kg. After haemodynamic stabilization the CB₁ receptor agonist CP-55,940 (0.1 mg/kg i. v.) was administered and the hypotensive effect established (Wagner, 2001).

### EXAMPLE 4

### PHARMACOLOGICAL TEST RESULTS

In the table below, the in vitro and in vivo pharmacological data obtained by the protocols given above are collected. The results of the compounds of the invention are compared with those of compounds disclosed in EP 1 713 475.

| | In vitro pharmacology: h-CB₁ receptor | | in vivo pharmacology |
|---|---|---|---|
| | receptor binding | functional activity | CB-agonist induced |
| | displacement | inhibition AA release | blood pressure |
| | [³H]-CP-55,940 | [³H]-Arachidonic acid | rat |
| compound | pKᵢ | pA₂ | ID₅₀ (mg/kg, p.o.) |
| EP 1 713 475 | | | |
| Compound 1 | | | =30 |
| Compound 2 | | | >30 |
| Compound 3 | 7.5 | | =31 |
| Compound 4 | 7.0 | | >30 |
| Compound 5 | | | >30 |
| Compound 6 | 7.6 | | |
| Compound 7 | | | >30 |
| Compound 8 | | | >30 |
| Compound 9 | | | >30 |
| Compound 10 | 7.1 | | >30 |
| Compound 11 | | | >30 |
| Present invention | | | |
| Compound 1 | 8.0 | 8.8 | 6 |
| racemate (1 + 2) | 7.6 | | 9 |
| Compound 2 | 6.6 | 7.1 | >30 |
| Compound 3 | 7.8 | | |
| Compound 4 | 8.5 | 9.5 | |

The affinity for human CB₁ receptors was found to be 25 fold higher for compound 1 (5R) than for its (5S)-enantiomer (compound 2). As antagonist (pA₂), compound 1 was found to be 40 times more potent than compound 2. In addition, compound 1 was found active after oral administration in the in vivo CB₁ mediated (CP-55,940-induced) hypotension test whereas compound 2 was found inactive.

It was found that the 1,3,5-trisubstituted 4,5-dihydro-1H-pyrazole derivatives exemplified in EP 1 713 475 (Compounds 1-11) showed poor activities in vivo in a CB₁-mechanistic pharmacological model after oral administration. In contrast, compound 1 and its racemate 1 which are representatives from the present invention both have potent activities in vivo after oral administration in this model.

### EXAMPLE 5

### PHARMACEUTICAL PREPARATIONS

For clinical use, compounds of formula (I) are formulated into pharmaceutical compositions that are important and novel embodiments of the invention because they contain the compounds, more particularly specific compounds disclosed herein. Types of pharmaceutical compositions that may be used include: tablets, chewable tablets, capsules (including microcapsules), solutions, parenteral solutions, ointments (creams and gels), suppositories, suspensions, and other types disclosed herein, or are apparent to a person skilled in the art from the specification and general knowledge in the art. The active ingredient for instance, may also be in the form of an inclusion complex in cyclodextrins, their ethers or their esters. The compositions are used for oral, intravenous, subcutaneous, tracheal, bronchial, intranasal, pulmonary, transdermal, buccal, rectal, parenteral or other ways to administer. The pharmaceutical formulation contains at least one compound of formula (I) in admixture with at least one pharmaceutically acceptable adjuvant, diluent and/or carrier. The total amount of active ingredients suitably is in the range of from about 0.1% (w/w) to about 95% (w/w) of the formulation, suitably from 0.5% to 50% (w/w) and preferably from 1% to 25% (w/w). In some embodiments, the amount of active ingredient is greater than about 95% (w/w) or less than about 0.1% (w/w).

The compounds of the invention can be brought into forms suitable for administration by means of usual processes using auxiliary substances such as liquid or solid, powdered ingredients, such as the pharmaceutically customary liquid or solid fillers and extenders, solvents, emulsifiers, lubricants, flavorings, colorings and/or buffer substances. Frequently used auxiliary substances include magnesium carbonate, titanium dioxide, lactose, saccharose, sorbitol, mannitol and other sugars or sugar alcohols, talc, lactoprotein, gelatin, starch, amylopectin, cellulose and its derivatives, animal and vegetable oils such as fish liver oil, sunflower, groundnut or sesame oil, polyethylene glycol and solvents such as, for example, sterile water and mono- or polyhydric alcohols such as glycerol, as well as with disintegrating agents and lubricating agents such as magnesium stearate, calcium stearate, sodium stearyl fumarate and polyethylene glycol waxes. The mixture may then be processed into granules or pressed into tablets. A tablet is prepared using the ingredients below:

| Ingredient | Quantity (mg/tablet) |
|---|---|
| COMPOUND No. 1 | 10 |
| Cellulose, microcrystalline | 200 |
| Silicon dioxide, fumed | 10 |
| Stearic acid | 10 |
| Total | 230 |

The components are blended and compressed to form tablets each weighing 230 mg.

The active ingredients may be separately premixed with the other non-active ingredients, before being mixed to form a formulation. The active ingredients may also be mixed with each other, before being mixed with the non-active ingredients to form a formulation.

Soft gelatin capsules may be prepared with capsules containing a mixture of the active ingredients of the invention, vegetable oil, fat, or other suitable vehicle for soft gelatin capsules. Hard gelatin capsules may contain granules of the active ingredients. Hard gelatin capsules may also contain the active ingredients together with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories containing the active substance mixed with a neutral fat base; (ii) in the form of a gelatin rectal capsule containing the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration.

Liquid preparations may be prepared in the form of syrups, elixirs, concentrated drops or suspensions, e.g. solutions or suspensions containing the active ingredients and the remainder consisting, for example, of sugar or sugar alcohols and a mixture of ethanol, water, glycerol, propylene glycol and polyethylene glycol. If desired, such liquid preparations may contain coloring agents, flavoring agents, preservatives, saccharine and carboxymethyl cellulose or other thickening agents. Liquid preparations may also be prepared in the form of a dry powder, reconstituted with a suitable solvent prior to use. Solutions for parenteral administration may be prepared as a solution of a formulation of the invention in a pharmaceutically acceptable solvent. These solutions may also contain stabilizing ingredients, preservatives and/or buffering ingredients. Solutions for parenteral administration may also be prepared as a dry preparation, reconstituted with a suitable solvent before use.

Also provided according to the present invention are formulations and ‘kits of parts’ comprising one or more containers filled with one or more of the ingredients of a pharmaceutical composition of the invention, for use in medical therapy. Associated with such container(s) can be various written materials such as instructions for use, or a notice in the form prescribed by a governmental agency regulating the manufacture, use or sale of pharmaceuticals products, noticing reflects approval by the agency of manufacture, use, or sale for human administration. The use of formulations of the invention in the manufacture of medicaments for use in the treatment of a condition wherein modulation of cannabinoid CB₁ receptors is required or desired, and methods of medical treatment or comprising the administration of a therapeutically effective total amount of at least one compound of formula (I), either as such or, in the case of prodrugs, after administration, to a patient suffering from, or susceptible to, a condition wherein modulation of cannabinoid CB₁ receptors is required or desired.

By way of example and not of limitation, several pharmaceutical compositions are given, comprising preferred active compounds for systemic use or topical application. Other compounds of the invention or combinations thereof, may be used in place of (or in addition to) said compounds. The concentration of the active ingredient may be varied over a wide range as discussed herein. The amounts and types of ingredients that may be included are well known in the art.

### BIBLIOGRAPHY

Adam, J. et al., Progress in Med. Chem. 2006, 44, 207-329; Eds. King and Lawton, Elsevier, Amsterdam
Berge, S. M.: “Pharmaceutical salts”, J. Pharmaceutical Science, 66, 1-19 (1977).
Bickel, M. H., “The pharmacology and Biochemistry of N-oxides”, Pharmacological Reviews, 21(4), 325-355, 1969.
Boyd, S. T. and Fremming, B. A. Ann. Pharmacother. 2005, 39, 684-690
Byrn et al., Pharmaceutical Research, 12(7), 945-954, 1995.
Carai, M. A. M. et al., Life Sc. 2005, 77, 2339-2350
Dwyer & Meilor,: “Chelating agents and Metal Chelates”, Academic Press, chapter 7, 1964.
Hertzog, D. L. Expert Opin. Ther. Patents, 14, 1435-1452, 2004
Hogenauer, E. K. Expert Opin. Ther. Patents 2007, 17, 1457.
Lange, J. H. M. and Kruse, C. G., C. Curr. Opin. Drug Discovery Dev, 7, 498-506, 2004
Lange, J. H. M. and Kruse, C. G. Drug Discov. Today, 10, 693-702, 2005
Martin, E. W. (Editor), “Remington: The Science and Practice of Pharmacy”, Mack Publishing Company, 19^{th} Edition, Easton, Pa., Vol 2., Chapter 83, 1447-1462, 1995.
Muccioli, G. G. et al., Curr. Med. Chem., 12, 1361-1394, 2005
Muccioli, G. G. and Lambert, D. M., Expert Opin. Ther. Patents, 16, 1405-1423, 2006
Padgett, L. W. Life Sc., 77, 1767-1798, 2005.
Reggio, P. H. Curr. Pharm. Des. 2003, 9, 1607-1633
Smith, R. A. and Fathi, Z. Idrugs, 8, 53-66, 2005.
Sorbera, L. A. et al. Drugs Fut. 2005, 30, 128-137
Spek, A. L., J. Appl. Cryst. 36 (2003) 7-13
Srivastava et al. (J. Med. Chem. 2007, 50, 5951-5966
Thakur, G. A. et al., Mini-Rev. Med. Chem., 5, 631-640, 2005.
Wagner, J. A., et al., Hemodynamic effects of cannabinoids: coronary and cerebral vasodilation mediated by cannabinoid CB₁ receptors. Eur. J. Pharmacol. 2001, 423, 203-10).

### CITED PATENTS AND PATENT APPLICATIONS

EP 1 713 475 (published as WO 2005/074920)
EP 1 743 892

1-10. (canceled)

11. The (5R)-enantiomer of a compound of formula (I): 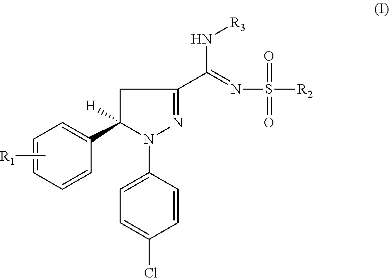
or a tautomer, stereoisomer, or N-oxide thereof, or a pharmacologically acceptable salt of any of the foregoing, wherein:
R₁ is chosen from a hydrogen and a fluoro atom;
R₂ is chosen from a piperidinyl and a pyrrolidinyl group, which may each be substituted with one or two fluoro atoms or a trifluoromethyl group; and
R₃ is chosen from a methyl and an ethyl group.

12. The compound as claimed in claim 11, wherein R₂ is a piperidin-1-yl group substituted at its 4-position with one or two fluoro atoms or a trifluoromethyl group, and R₃ is a methyl group.

13. The compound as claimed in claim 11, wherein the compound is chosen from:
(5R)-N-[(4,4-difluoropiperid in-1-yl)sulfonyl]-N′-methyl-1-(4-chlorophenyl)-5-phenyl-4,5-dihydro-(1H)-pyrazole-3-carboxamidine;
(5R)-N-[(4-fluoropiperidin-1-yl)sulfonyl]-N′-methyl-1-(4-chlorophenyl)-5-phenyl-4,5-dihydro-(1H)-pyrazole-3-carboxamidine; and
(5R)-N-[(4-(trifluoromethyl)piperidin-1-yl)sulfonyl]-N′-methyl-1-(4-chlorophenyl)-5-phenyl-4,5-dihydro-(1H)-pyrazole-3-carboxamidine.

14. A method for treating at least one disorder or condition chosen from psychosis, anxiety, depression, attention deficits, memory disorders, cognitive disorders, appetite disorders, obesity, juvenile obesity, drug induced obesity, addiction, drug dependence, neurological disorders, neurodegenerative disorders, dementia, traumatic brain injury, stroke, Parkinson's disease, Alzheimer's disease, neuroinflammatory disorders, septic shock, cancer, diabetes, asthma, respiratory diseases, gastrointestinal disorders, liver cirrhosis, arthritis, gastric ulcers, diarrhoea and cardiovascular disorders, the method comprising administering a pharmaceutical composition to a patient in need thereof, wherein said composition comprises a compound of formula (I): 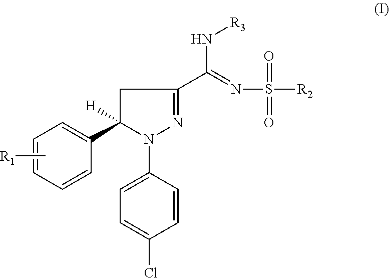
or a tautomer, stereoisomer, or N-oxide thereof, or a pharmacologically acceptable salt of any of the foregoing, wherein:
R₁ is chosen from a hydrogen and a fluoro atom;
R₂ is chosen from a piperidinyl and a pyrrolidinyl group, which may each be substituted with one or two fluoro atoms or a trifluoromethyl group; and
R₃ is chosen from a methyl and an ethyl group.

15. A method for preparing a pharmaceutical composition, the method comprising combining at least one pharmaceutically acceptable carrier, at least one pharmaceutically acceptable auxiliary substance, or a combination thereof, with a compound of formula (I): 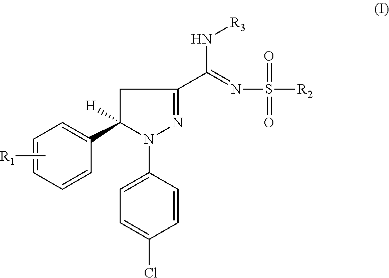
or a tautomer, stereoisomer, or N-oxide thereof, or a pharmacologically acceptable salt of any of the foregoing, wherein:
R₁ is chosen from a hydrogen and a fluoro atom;
R₂ is chosen from a piperidinyl and a pyrrolidinyl group, which may each be substituted with one or two fluoro atoms or a trifluoromethyl group; and
R₃ is chosen from a methyl and an ethyl group.

16. A pharmaceutical composition comprising at least one pharmaceutically acceptable carrier, at least one pharmaceutically acceptable auxiliary substance, or combination thereof, and a pharmacologically active amount of at least one compound of formula (I): 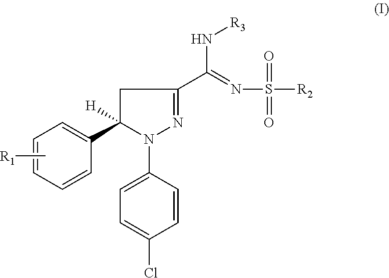
or at least one pharmacologically acceptable salt thereof, wherein:
R₁ is chosen from a hydrogen and a fluoro atom;
R₂ is chosen from a piperidinyl and a pyrrolidinyl group, which may each be substituted with one or two fluoro atoms or a trifluoromethyl group; and
R₃ is chosen from a methyl and an ethyl group.

17. The pharmaceutical composition according to claim 16, wherein the composition further comprises at least one additional therapeutic agent.

18. A process for preparing a compound of formula (I): 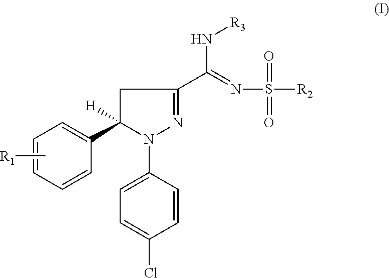
or a tautomer, stereoisomer, or N-oxide thereof, or a pharmacologically acceptable salt of any of the foregoing, wherein:
R₁ is chosen from a hydrogen and a fluoro atom;
R₂ is chosen from a piperidinyl and a pyrrolidinyl group, which may each be substituted with one or two fluoro atoms or a trifluoromethyl group; and
R₃ is chosen from a methyl and ethyl group;
the process comprising:
(i) reacting a carbonyl chloride of formula (IV): 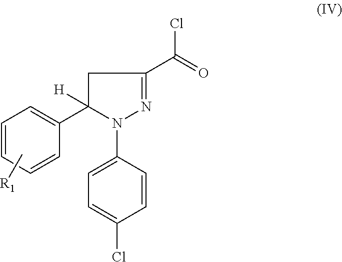
with a compound having formula R₂SO₂NH₂, in the presence of a base, to yield a compound of formula (V): 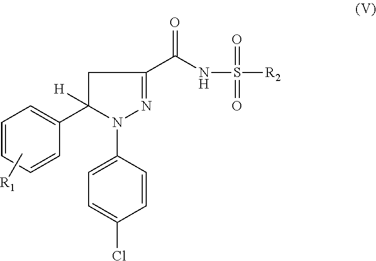
(ii) reacting a compound of formula (V) with a chlorinating agent to yield a compound of formula (VI): 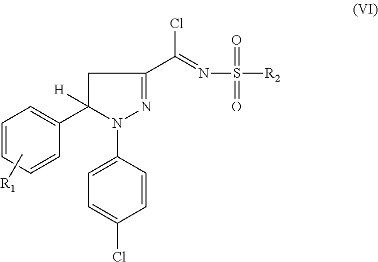
(iii) reacting a compound of formula (VI) with an amine of formula NH₂R₃ to yield a compound of formula (VII): 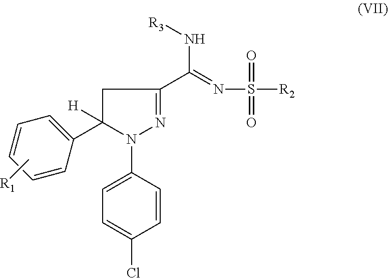
(iv) separating a racemate of formula (VII) into its two enantiomers by preparative HPLC; and
(v) determining the compound of formula (I), wherein C₅ of its 4,5-dihydropyrazole moiety has the R configuration.

19. The process as claimed in claim 18, wherein in step (ii) the chlorinating agent is POCl₃.

20. The process as claimed in claim 18, wherein the reaction in step (ii) is carried out in the presence of 4-dimethylaminopyridine.

21. A compound of formula (I*), 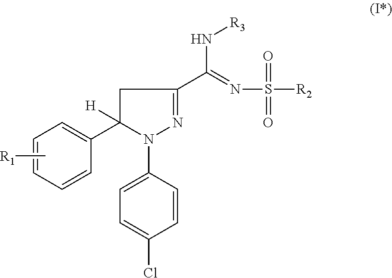
wherein:
R₁ is chosen from a hydrogen and a fluoro atom;
R₂ is chosen from a piperidinyl and a pyrrolidinyl group, which may each be substituted with one or two fluoro atoms or a trifluoromethyl group; and
R₃ is chosen from a methyl and an ethyl group; and
wherein the compound of formula (I*) is chosen from:
N-[(4,4-difluoropiperid in-1-yl)sulfonyl]-N′-methyl-1-(4-chlorophenyl)-5-phenyl-4,5-dihydro-(1H)-pyrazole-3-carboxamidine;
N-[(4,4-difluoropiperidin-1-yl)sulfonyl]-N′-methyl-1-(4-chlorophenyl)-5-(2-fluorophenyl)-4,5-dihydro-(1H)-pyrazole-3-carboxamidine;
N-[(4-(trifluoromethyl)piperidin-1-yl)sulfonyl]-N′-methyl-1-(4-chlorophenyl)-5-phenyl-4,5-dihydro-(1H)-pyrazole-3-carboxamidine;
N-[(4-fluoropiperidin-1-yl)sulfonyl]-N′-methyl-1-(4-chlorophenyl)-5-phenyl-4,5-dihydro-(1H)-pyrazole-3-carboxamidine;
N-[(3-fluoropiperidin-1-yl)sulfonyl]-N′-methyl-1-(4-chlorophenyl)-5-phenyl-4,5-dihydro-(1H)-pyrazole-3-carboxamidine;
N-[(3,3-difluoropiperidin-1-yl)sulfonyl]-N′-methyl-1-(4-chlorophenyl)-5-phenyl-4,5-dihydro-(1H)-pyrazole-3-carboxamidine;
N-[(3,3-difluoropyrrolidin-1-yl)sulfonyl]-N′-methyl-1-(4-chlorophenyl)-5-phenyl-4,5-dihydro-(1H)-pyrazole-3-carboxamidine;
N-[(4,4-difluoropiperidin-1-yl)sulfonyl]-N′-ethyl-1-(4-chlorophenyl)-5-phenyl-4,5-dihydro-(1H)-pyrazole-3-carboxamidine; and
N-[(4-fluoropiperidin-1-yl)sulfonyl]-N′-ethyl-1-(4-chlorophenyl)-5-phenyl-4,5-dihydro-(1H)-pyrazole-3-carboxamidine.
